(19) United States
(12) Patent Application Publication
(10) Pub. No.: US 2011/0065165 A1
(43) Pub. Date: Mar. 17, 2011

(54) METHOD FOR CULTURING PLANKTONIC MICROALGAE

(75) Inventors: Masumi TAKEBE, Hamamatsu-shi (JP); Hiroshi Tsuchiya, Hamamatsu-shi (JP)

(73) Assignee: HAMAMATSU PHOTONICS K.K., Hamamatsu-shi (JP)

(21) Appl. No.: 12/878,285

(22) Filed: Sep. 9, 2010

(30) Foreign Application Priority Data
Sep. 16, 2009 (JP) 2009-214832

(51) Int. Cl. C12N 1/12

(52) U.S. Cl. 435/257.1

(57) ABSTRACT

The present invention relates to a method for culturing planktonic microalgae, comprising: culturing planktonic microalgae with a culture substrate containing a visible light transmissive fiber with a hydrophilic surface, where a space diameter between the fibers is from 10 μm to 500 μm.

### BACKGROUND OF THE INVENTION

1. Field of the Invention

The present invention relates to a method for culturing planktonic microalgae.

2. Related Background Art

Microalgae have been utilized in various industrial fields because they grow rapidly and have high photosynthetic capacities. For example, extracting useful materials from microalgae and using them as raw materials for foods, pharmaceuticals, feeds, fertilizers, and the like are practiced. In addition, since microalgae have high photosynthetic capacities, i.e., carbon dioxide fixation capacities, culturing them as a warming countermeasure is also useful. Further, recently, producing petroleum and bioethanol by utilizing carbohydrates and lipids that microalgae accumulate in their cells has been receiving attention. Therefore, a technique for mass-culturing microalgae is required.

For mass-culturing microalgae, a culture vessel of large size such as a tank or a pool is commonly used. As a method for culturing microalgae, for example, in Japanese Patent Application Laid-Open Publication No. 2007-330215, a method of using a culture tool which, to prevent germ contamination, places a liquid and algae in a reservoir composed of a non-porous hydrophilic film and cultures them therein, and which takes nutrient salts or the like from a liquid outside of the reservoir, is disclosed. In Japanese Patent Application Laid-Open Publication No. 05-64577, a plant factory which combines cultivation of vegetables and culturing of edible algae is disclosed, and the factory utilizes used waste liquid that has been used in the cultivation of vegetables, as a culture liquid for the edible algae.

### SUMMARY OF THE INVENTION

Among microalgae, planktonic microalgae are useful since they are used as feeds for shellfish or fish. Planktonic microalgae are algae which have a flagellum and/or cilia and live moving freely in liquid, and they tend not to cluster fat to disperse uniformly; even when they aggregate in water, they fall in water as aggregated masses and after falling they dissociate and disperse again in water. Accordingly, when mass-culturing planktonic microalgae in a culture vessel of large size such as a tank or a pool using a conventional method for culturing planktonic microalgae, it has been necessary to recover the whole culture liquid in the culture vessel to recover the cultured planktonic microalgae since planktonic microalgae drift in the culture vessel and disperse uniformly in the culture liquid, which has been huge by costly and laborious.

Thus, the present invention aims to provide a method for culturing planktonic microalgae which method allows easy recovery of planktonic microalgae that disperse uniformly.

The present invention provides a method for culturing planktonic microalgae, comprising: culturing planktonic microalgae with a culture substrate containing a visible light transmissive fiber with a hydrophilic surface, where a space diameter between the fibers is from 10 μm to 500 μm.

When culturing planktonic microalgae with a culture substrate containing a visible light transmissive fiber with a hydrophilic surface, where a space diameter between the fibers is in the above-described range, because the planktonic microalgae cluster locally and densely on the surface or inside of the culture substrate, the planktonic microalgae can be easily recovered in large quantities by recovering the clustered part together with the culture substrate.

It is preferred that the fiber described above is rock wool. Since rock wool has suitable water retention capacities, suitable water absorbency, and suitable light transmission properties, planktonic microalgae are cultured more easily and can be mass-cultured.

It is preferable to perform the above-described method for culturing planktonic microalgae utilizing nitrogen in the air as a nutrient source using a nitrogen-fixing photocatalyst. By utilizing nitrogen in the air as a nutrient source, one can reduce the nutrient source in the culture liquid, and can reduce the cost, and in addition, the disposal of the waste liquid becomes easy.

According to the culture method of the present invention, since planktonic microalgae cluster densely on the culture substrate one can recover planktonic microalgae easily.

### BRIEF DESCRIPTION OF THE DRAWINGS

FIG. 1 is a diagram which shows a culture device for planktonic microalgae;

FIG. 2 is a diagram of culturing using a plurality of culture devices;

FIG. 3 is a diagram of culturing of planktonic microalgae in a culture room using a plurality of culture devices;

FIG. 4 is a photograph which represents Euglena on the first day of culturing;

FIG. 5 is a photograph which represents Chlamydomonas on the first day of culturing;

FIG. 6 is a photograph which represents Euglena after 4 days of culture;

FIG. 7 is a photograph which represents Chlamydomonas after 4 days of culture; and

FIG. 8 is a photograph (A) of culturing Chlamydomonas in experimental medium after 4 days of culture and a photograph (B) of 0.03 mm grid photographed at the same magnification as in the photograph (A).

1 Light source
2 Light ray
3 Planktonic microalgae
4 Culture substrate
5 Nitrogen-fixing photocatalyst
10, 20 Culture device
11 Sun
12 Optical guiding device
100 Culture room

### DESCRIPTION OF THE PREFERRED EMBODIMENTS

The preferred embodiments of the present invention will now be described with reference to the drawings.

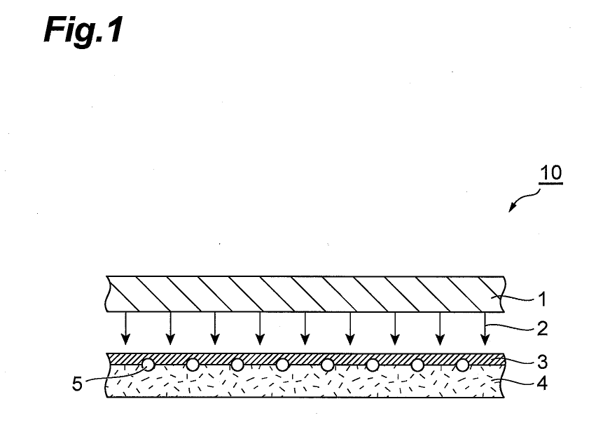

FIG. 1 is a diagram which shows a culture device 10 for planktonic microalgae used in the culture method of the present invention. The culture device 10 is a device used in culture of planktonic microalgae. The culture device 10 is equipped with a light source 1 and a culture substrate 4, and planktonic microalgae 3 are cultured as clustered locally on a surface or inside of the culture substrate 4 on which a light ray 2 from the light source 1 is emitted. The culture substrate 4 is provided with a nitrogen-fixing photocatalyst 5 on its surface.

When the light ray 2 from the light source 1 is emitted on the culture substrate 4, the planktonic microalgae 3 existing on the surface or inside of the culture substrate 4 photosynthesize and obtain a nutrient source from the culture liquid permeated in the culture substrate 4 to grow, thereby being cultured. By recovering the planktonic microalgae 3 cultured as clustered locally in part of the culture substrate 4 together with all of the culture substrate 4 or with the part thereof at which the planktonic microalgae 3 cluster from the culture device 10 and separating them from the culture substrate 4 by filtration, centrifugation, or other appropriate methods, it is possible to recover the cultured planktonic microalgae 3 in large quantities.

The culture substrate 4 contains a visible light transmissive fiber with hydrophilic surface, and a space diameter between the fibers is from 10 μm to 500 μm. With this range of space diameters, the culture liquid moderately fills the gaps between the fibers; therefore, the planktonic microalgae can swim freely between the fibers, thereby allowing them to obtain a nutrient source to grow. In addition, with this range of space diameters, oxygen and carbon dioxide are moderately supplied; therefore, the planktonic microalgae respire and photosynthesize easily, thereby allowing them to grow. Further, with this range of space diameters, the planktonic microalgae are kept dense between the fibers even when they aggregate into masses; therefore, the problem will not arise in that, when cultured in a culture vessel of large size, the planktonic microalgae fall in water as masses and after falling they disperse uniformly. The space diameter described above is, more preferably, from 20 μm to 200 μm, and still more preferably, from 30 μm to 100 μm. This is because the planktonic microalgae are maintained more easily and the planktonic microalgae grow more easily.

The fiber with a hydrophilic surface, which is used in the culture substrate 4, refers to a fiber that is hydrophilic itself and to a fiber whose surface is hydrophilic by applying a hydrophilic processing to the surface of the fiber. Examples of a fiber that is hydrophilic itself which can be used include, for example, regardless of whether it is natural fiber or artificial fiber, hydrophilic fibers such as mineral fibers such as rock wool, asbestos; vinylo; vinylal; acetate; rayon; cupra; cotton; hemp; wool; silk. Because the culture substrate 4, which contains such fibers, is highly hygroscopic and water-retentive, it absorbs culture liquid well and retains them between the fibers, and as a result, an environment in which planktonic microalgae grow and grow easily is established. Moreover, if the fiber itself is hydrophilic, the work of applying a hydrophilic processing to the surface of the fiber can be omitted.

Examples of a fiber whose surface can be hydrophilic by applying a hydrophilic processing to the surface of the fiber and which can be used include, for example, hydrophobic fibers such as glass fibers such as glass wool; polypropylene; polyamides such as nylon; polyesters; polyolefins; acrylic fibers; polyethylene and triacetate. These hydrophobic fibers can be hydrophilic at the surface by applying a hydrophilic processing, for example, introducing hydrophilic groups on the surface of the fiber, making the surface of the fiber porous, and applying a coating processing to the surface of the fiber. The hydrophilic processing may be applied to the entire surface of the hydrophobic fiber or to part of the surface of the hydrophobic fiber. Since these fibers are hydrophilic at the surface by applying a hydrophilic processing to them, as with the above-mentioned hydrophilic fibers, they are highly hygroscopic and water-retentive, and establish an environment in which planktonic microalgae grow and multiply easily. In addition, since these hydrophobic fibers have a high strength, the strength of a culture substrate comprising these hydrophobic fibers is also high; therefore, in separating the planktonic microalgae from the culture substrate, the planktonic microalgae can be effectively separated by applying great mechanical pressure to the culture substrate. Further, by applying a coating processing to the surface as a way of hydrophilic processing, it becomes easy to adjust the space diameter between the fibers according to the size of the planktonic microalgae. The hydrophilic processing can be applied not only to the hydrophobic fibers but also to the entire surface or part of the surface of the above-mentioned hydrophilic fibers; they can also enhance the hygroscopicity and water retention capacities, and make it easy to adjust the space diameter between the fibers.

Among the fibers with hydrophilic surface, it is preferable to use rock wool. When rock wool is used, recovery of planktonic microalgae becomes extremely easy because the planktonic microalgae cluster more densely at local on the surface of or inside the culture substrate.

The fibers contained in the culture substrate 4 are visible light transmissive. Being visible light transmissive means that the visible light transmission rate of the fiber is high, that is, visible light, light with a wavelength of 380 nm to 750 nm, is transmitted sufficiently for the planktonic microalgae to photosynthesize. The visible light transmission rate can be determined by a method which is based on spectrophotometry. In the present invention, the visible light transmission rate of the fiber, in the case of 5 mm thickness, is not less than 30%, preferably not less than 50%, and more preferably not less than 70%. The planktonic microalgae 3 are cultured mainly on the surface of the culture substrate 4 on which the light ray 2 is emitted, but the planktonic microalgae 3 can also be cultured inside the culture substrate 4 because the fiber contained in the culture substrate 4 are visible light transmissive and visible light reaches into the culture substrate 4.

As long as the space diameter between the fibers is from 10 μm to 500 μm and the culture substrate 4 contains a visible light transmissive fiber with hydrophilic surface, the culture substrate 4 may be in the form of either nonwoven fabric or woven fabric, which can take the shape of film, sheet, granule, sponge, and rope, and can be used alone or in combination by, for example, laminating or combining them.

In cases where the culture substrate 4 is in the form of sheet, the thickness of the sheet is preferably from 1 mm to 20 mm, more preferably from 3 mm to 15 mm, and still more preferably from 5 mm to 10 mm. In cases where the culture substrate 4 is in the form of granule, if the maximum diameter of a granule is particle size, the average particle size is preferably from 1 mm to 20 mm, more preferably from 3 mm to 15 mm, and still more preferably from 5 mm to 10 mm.

Besides the visible light transmissive fiber with hydrophilic surface, a variety of materials may be contained in the culture substrate 4. For example, the culture substrate 4 may contain materials which can be a nutrient source for planktonic microalgae, including nitrogen, phosphate, potassium, calcium, magnesium, sulfur, iron, manganese, zinc, copper, boron, molybdenum, sodium, aluminum, cobalt, and silicic acid. The culture substrate 4 may also contain a watering agent, water retention agent, chelate compound, surfactant, strengthening agent and the like.

As a strengthening agent, materials such as a piece of fiber, a piece of paper, a piece of metal, a piece of plastic, a piece of ceramic, a piece of wood and the like can be used, and the culture substrate 4 can be formed by mixing the above-described visible light transmissive fiber with hydrophilic surface and the strengthening agent.

Further, in consideration of the strength, handling and the like of the culture substrate 4, the culture substrate 4 formed may be combined with a support and used in the culture device 10. The support may be combined by, for example, laminating or pasting it on the culture substrate 4. In laminating or pasting the support on the culture substrate 4 in the form of sheet, it is preferable to laminate or paste supporting materials on the opposite side of the side of the culture substrate 4 on which the light ray 2 is emitted. Examples of the materials for the support include hard materials such as metal, plastic, ceramic, and wood.

On the surface of the culture substrate 4 and between the fibers, the planktonic microalgae 3 are cultured. “Planktonic microalgae,” as used in the present invention, refers to microalgae that are capable of swimming in liquid because they have organs and components necessary for migration, such as flagellum and/or cilium, and viscous materials. Examples of the planktonic microalgae cultured in the present invention include golden-brown algae (Dinobryon, Ochromonas, Mallomonas, Synura), Raphidophyceae (Gonyostomum, Vacuolaria, Merotricha), diatom (Thalassiosira, Coscinodiscus, Biddulphia, Chaetoceros, Fragilaria, Coscinodiscus), Chrysophyceae (Uroglena), euglenids (Euglena, Phacus, Monomorphina, Trachelomonas), Cryptophyceae (Rhodomonas), dinoflagellate (Noctiluca, Prorocentrum, Dinophysis, Gymnodinium, Peridinium, Gonyaulax, Dhinococcales), cryptophyte algae (Cryptomonas, Chroomonas), Haptophyceae (Pavlova, Phaeocystis, Prymnesium, Isochrysis), green algae (Chlamydomonas, Chlorogonium, Haematococcus, Carteria, Volvox, Pleodorina, Gonium, Tetrabaena, Botryococcus) and the like.

The planktonic microalgae cultured at the surface of the culture substrate 4 or in the culture substrate 4 can be recovered as separated from the culture substrate 4 by recovering them together with all of the culture substrate 4 or with the part thereof on which the planktonic microalgae locally cluster, followed by, for example, filtering and centrifuging the culture substrate 4. For some application of the planktonic microalgae, for example, for use as fertilizers, the planktonic microalgae can be utilized without being separated from the culture substrate 4 but together with the culture substrate 4. The part on which the planktonic microalgae locally cluster in the culture substrate 4 can be easily distinguished because the planktonic microalgae have a green color.

The light source 1 is a light source such as a natural light source, for example, sun, or a artificial light source, or is an optical guiding device that guides a light from the natural light source or the artificial light source, and it emits the light ray 2, which is effective for the photosynthesis and the multiplication of the planktonic microalgae. Examples of the light source 1 include, for example, sun, incandescent lamp, fluorescent lamp, sodium lamp, metal halide lamp, high-pressure sodium lamp, light-emitting diode, laser diode, light-emitting panel, and optical guiding panel.

The light ray 2, which is a light effective for the photosynthesis and multiplication of the planktonic microalgae, is emitted from the light source 1 and emitted on the culture substrate 4. The light ray 2 preferably is emitted such that the illuminance of the light that is emitted on the culture substrate 4 is from 10 to 1000 μE·m⁻²·s⁻¹. The light ray 2, as a light effective for the photosynthesis and multiplication of the planktonic microalgae, preferably is a light comprising a light with a wavelength of 380 nm to 750 nm.

The culture substrate 4 is provided with the nitrogen-fixing photocatalyst 5. The nitrogen-fixing photocatalyst 5 is formed from titanium oxide or the like, and fixes gaseous nitrogen, such as nitrogen in the air, nitrogen monoxide, and nitrogen dioxide, into a form that the planktonic microalgae can utilize as a nutrient source, such as ammonia nitrogen or nitrate nitrogen. The nitrogen-fixing photocatalyst 5 can be placed on the surface or inside of the culture substrate 4. The culture device 10 may or may not have the nitrogen-fixing photocatalyst 5.

The culture substrate 4 is supplied with a culture liquid. With respect to the culture liquid, the culture conditions and the like in the culture method of the present invention, the culture liquid, the culture conditions and the like for the planktonic microalgae used in the general tank culture or pool culture can be used. For example, the temperature for culturing the planktonic microalgae can be from 5 to 40° C., and the humidity can be from 30% to 100%. Examples of the culture liquid used include Cramer-Myers medium, C medium, Koren-Hutner medium, Hutner medium, Euglena medium, TAP medium, MAF-6 medium, f/2 medium, CSi medium, Allen medium, BG-11 medium, CA medium, CAM medium, CB medium, CT medium, CYT medium, HUT medium, MBM medium, MDM medium, MG medium, P35 medium, Pro medium, SOT medium, SW medium, URO medium, VT medium and the like. As a method for supplying the culture liquid to the culture substrate 4, any method such as the method in which the culture substrate 4 is placed in a vessel filled with the culture liquid and the method in which the culture liquid is sprayed on the culture substrate 4, may be used as long as it is a method in which the culture liquid is permeated into the culture substrate 4. The culture liquid may be added so that the culture substrate 4 is completely immersed or may be added so that part of the culture substrate 4 is not immersed in the culture liquid and exposed to the air. In the latter case, the inside the culture substrate 4 exposed to the air is filled with the culture liquid by capillary action; the planktonic microalgae can grow there and, moreover, the planktonic microalgae can be cultured more densely than in the former case. Preferably, the air that contains carbon dioxide at a concentration of 0 to 50% is bubbled through the culture liquid with a ventilation volume of 0.01-1 vvm.

The culture device 10 in whole can be placed upright with the side of the culture substrate 4 on which side the light ray 2 is emitted perpendicular to the ground. The culture device 10 can also be tilted and leaned against the wall. By placing it upright or at a tilt, it is possible to culture even in a small space.

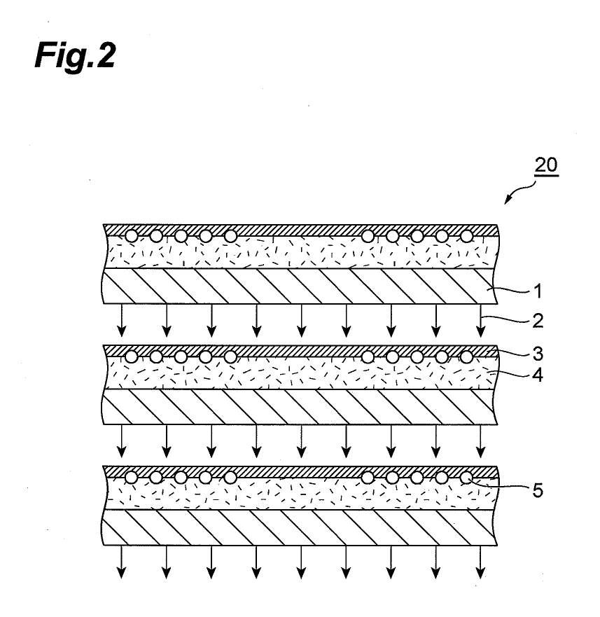

Although it is possible to culture planktonic microalgae with the culture device 10 alone, it is also possible to culture planktonic microalgae in multi-tiers in combination of a plurality of culture devices 10. FIG. 2 is a diagram which shows a culture device 20 for planktonic microalgae in which a plurality of culture devices 10 are stacked. By culturing in multi-tiers, it is possible to culture planktonic microalgae efficiently and in large quantities while saving space. In addition, in multi-tier culture, by using an optical guiding device, light can be guided to the culture substrate in the tier where the light from the light source does not reach directly, thereby space can be saved. Further, in multi-tier culture, the culture liquid can be circulated through the plurality of tiers.

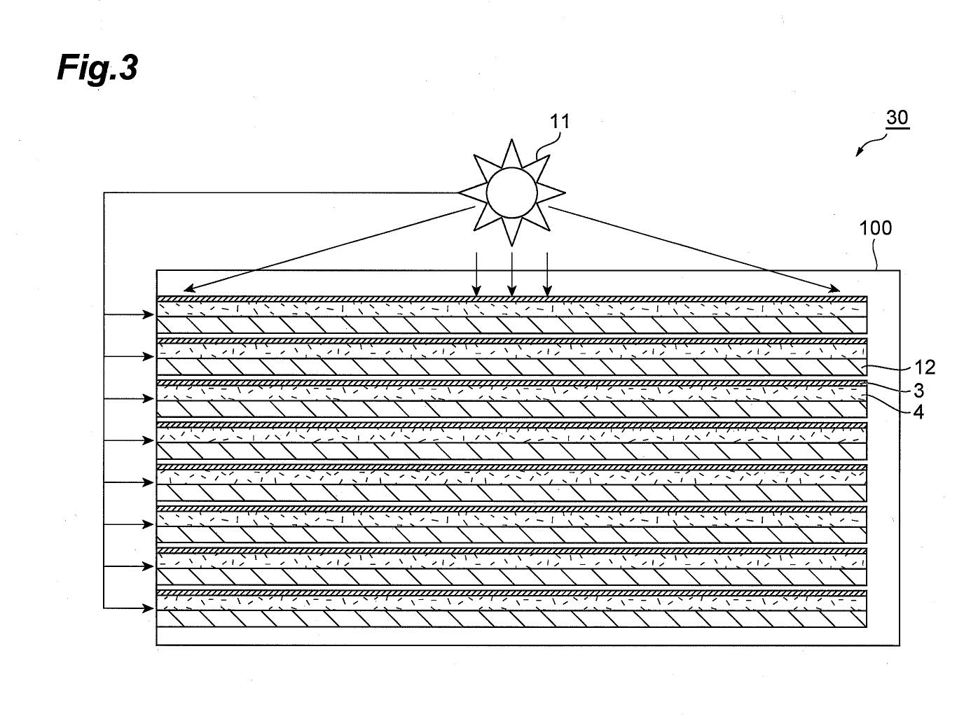

FIG. 3 shows how the culture is performed in a culture room using sunlight and an optical guiding device according to the culture method of the present invention. The light from sunlight 11 reaches the culture substrate 4 through an optical guiding device 12 of the culture device that is installed in a culture room 100. The planktonic microalgae 3 existing on the surface or inside of the culture substrate 4 photosynthesize using the guided light and they are cultured by obtaining a nutrient source from the culture liquid permeated in the culture substrate 4. The planktonic microalgae 3 cultured as clustered locally at part of the culture substrate 4 are recovered together with all of the culture substrate 4 or with the part thereof at which the planktonic microalgae cluster, and subsequently separated from the culture substrate 4 to be recovered.

### EXAMPLE

The present invention will now be described in more detail by way of the following example, but the present invention is not limited to these examples.

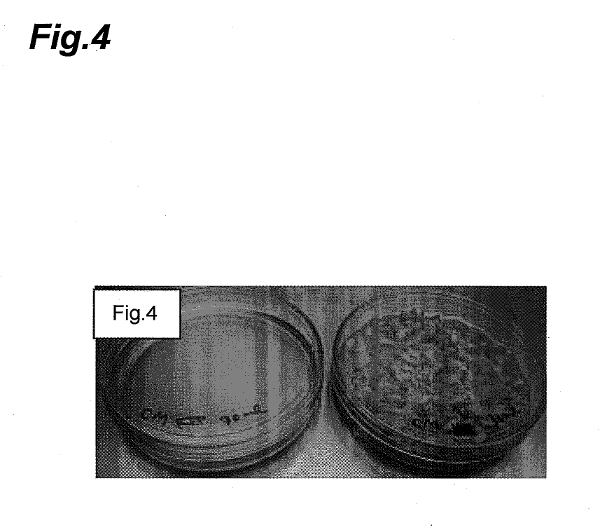

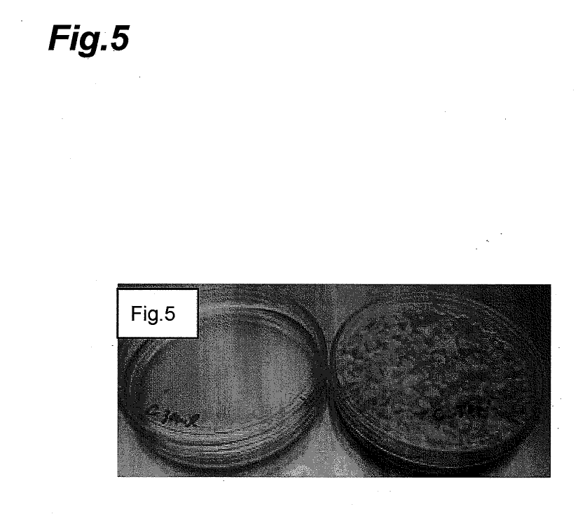

As planktonic microalgae, Euglena (Euglena gracilis strain Z) and Chlamydomonas (Chlamydomonas reinhardtii NIES-2235), unicellular flagellatae, were used. As a culture substrate, granulous rock wool was used. Space diameters of the rock wool are 10 μm to 500 μm and the average particle size is 4 mm. A liquid medium was prepared from Cramer-Myers medium and C medium. Granulous rock wool 2 g was placed in a plastic petri dish, which was then filled with 30 ml of the liquid medium to provide an experimental medium. Without placing rock wool in, 30 ml of the liquid medium alone was placed in a plastic petri dish to provide a control medium. The culture of Euglena and Chlamydomonas was started in the experimental medium and the control medium, respectively. The experiments were performed twice, and Euglena was added at the beginning of the culture to the experimental medium and the control medium such that the number of cells was, for the first experiment, 40±8 cells/μL, and for the second experiment, 15±3 cells/μL. Chlamydomonas was added to the experimental medium and the control medium such that the number of cells was, for the first experiment, 45±9 cells/μL, and for the second experiment, 60±10 cells/μL. The photographs of Euglena and Chlamydomonas at the beginning of the first culture are shown in FIG. 4 and FIG. 5, respectively. The left shows the control medium; the right shows the experimental medium.

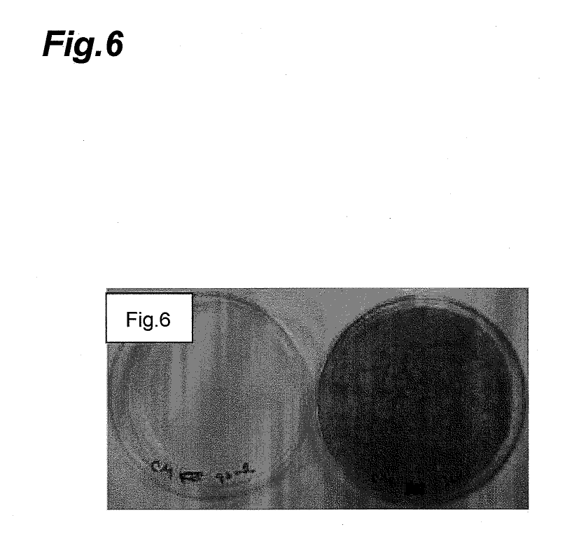

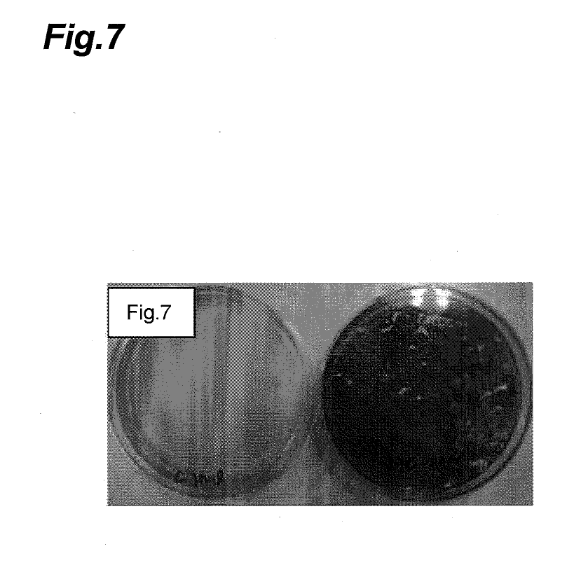

The photographs of Euglena and Chlamydomonas after 4 days from the start of the culture (the first experiment) are shown in FIG. 6 and FIG. 7. The left shows the control medium; the right shows the experimental medium. Deep color in the media indicates that the planktonic microalgae actively multiplied. Euglena and Chlamydomonas accumulated especially on the part of the rock wool which part was above the surface of the culture liquid and exposed to the air, and on which part the light struck well.

The number of cells of Euglena and that of Chlamydomonas in the experimental medium and the control medium after 4 days from the start of the culture were compared. From the control medium, 200 μL was collected; from the experimental medium, among the granulous rock wool, 10 granules that was above the liquid surface and had a deeper green color were collected (equivalent to a volume of 200 μL), and the number of cells of Euglena and Chlamydomonas was counted. The cell number per volume in each medium is shown in Table 1.

| | | Cell number | Cell number |
|---|---|---|---|
| | | in the first | in the second |
| | | experiment | experiment |
| | | (Cells/μL) | (Cells/μL) |
| Euglena | Experimental | 601.6 ± 128.6 | 141.6 ± 11.2 |
| | medium | | |
| | Control | 334.0 ± 23.9 | 78.0 ± 12.7 |
| | medium | | |
| Chlamydomonas | Experimental | 658.0 ± 90.4 | 1905 ± 240.1 |
| | medium | | |
| | Control | 126.4 ± 38.4 | 244.8 ± 30.7 |
| | medium | | |

Comparing the experimental medium with the control medium, almost equivalent results for the first and second experiment were obtained. That is, in the experimental medium, after 4 days of the culture, for Euglena, the cell number increased to 1.8 times that in the control; for Chlamydomonas, the cell number increased to 5.2-7.8 times that in the control. The reason why the difference occurred between the data of the first and second experiments is that the cell number at the beginning of the culture differs between the first and second experiments.

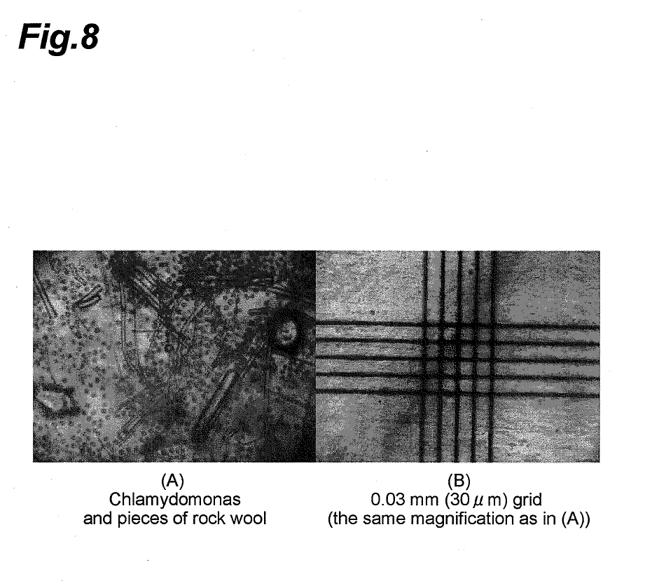

The photograph (A) of culturing Chlamydomonas in the experimental medium after 4 days of culture and the photograph (B) of 0.03 mm grid photographed at the same magnification as in the photograph (A) are shown in FIG. 8. In the photograph (A) of FIG. 8, the round granulous materials are Chlamydomonas, and the fibrous materials are rock wool. A number of the cells of Chlamydomonas are distributed between the fibers of the rock wool. When Chlamydomonas and Euglena were cultured using rock wool as a culture substrate, Chlamydomonas and Euglena had a tendency not to disperse uniformly in the culture liquid and to accumulate locally and densely at the surface of and in the rock wool.

According to the culture method of the present invention, since planktonic microalgae do not disperse uniformly and it is possible to culture the planktonic microalgae as clustered locally on a culture substrate, large quantities of the planktonic microalgae can be conveniently recovered by recovering only the part of the culture substrate on which the planktonic microalgae locally cluster even when they are muss-cultured. After recovering the culture substrate and the planktonic microalgae together, another culture can be started by placing a new culture substrate in the residual liquid medium. Further, by arranging culture substrates in multi-tiers, the culture can be carried out efficiently and in large quantities.

What is claimed is:

1. A method for culturing planktonic microalgae, comprising: culturing planktonic microalgae with a culture substrate containing a visible light transmissive fiber with a hydrophilic surface, where a space diameter between the fibers is from 10 μm to 500 μm.

2. The method according to claim 1, wherein the fiber is rock wool.

3. The method according to claim 1, wherein the method utilizing nitrogen in the air as a nutrient source using a nitrogen-fixing photocatalyst.

4. The method according to claim 2, wherein the method utilizing nitrogen in the air as a nutrient source using a nitrogen-fixing photocatalyst.
